(19) United States
(12) Patent Application Publication
(10) Pub. No.: US 2021/0169788 A1
(43) Pub. Date: Jun. 10, 2021

(54) ORAL PRODUCT AND METHOD OF MANUFACTURE

(71) Applicant: NICOVENTURES TRADING LIMITED, London (GB)

(72) Inventors: John Paul Mua, Advance, NC (US); John E Bunch, Cary, NC (US); Anthony Richard Gerardi, Winston-Salem, NC (US); Darrell Eugene Holton, JR., Clemmons, NC (US); Ronald K. Hutchens, East Bend, NC (US); Luis Monsalud, Kernersville, NC (US); Thomas H. Poole, Winston-Salem, NC (US); Frank Kelley St. Charles, Bowling Green, KY (US)

(21) Appl. No.: 16/707,580

(22) Filed: Dec. 9, 2019

(51) Int. Cl. A61K 9/00; A24B 15/16; A24B 15/40; A24B 15/38; A24B 13/00; A61K 47/38; A61K 47/36; A61K 47/10

(52) U.S. Cl. CPC A61K 9/0056; A24B 15/16; A24B 15/403; A61K 47/10; A24B 13/00; A61K 47/38; A61K 47/36; A24B 15/385

(57) ABSTRACT

A composition is provided, the composition including a binder system in an amount of at least about 5% by weight, based on the total weight of the composition, and at least one active ingredient and/or flavoring agent. The binder system includes at least one thermoplastic binding material and at least one plasticizer. The composition is typically substantially free of unbleached tobacco material, excluding any nicotine component present.

### FIELD OF THE DISCLOSURE

The present disclosure relates to flavored products intended for human use. The products are configured for oral use and deliver substances such as flavors and/or active ingredients during use. Such products may include tobacco or a product derived from tobacco, or may be tobacco-free alternatives.

### BACKGROUND

Tobacco may be enjoyed in a so-called “smokeless” form. Particularly popular smokeless tobacco products are employed by inserting some form of processed tobacco or tobacco-containing formulation into the mouth of the user. Conventional formats for such smokeless tobacco products include moist snuff, snus, and chewing tobacco, which are typically formed almost entirely of particulate, granular, or shredded tobacco, and which are either portioned by the user or presented to the user in individual portions, such as in single-use pouches or sachets. Other traditional forms of smokeless products include compressed or agglomerated forms, such as plugs, tablets, or pellets. Alternative product formats, such as tobacco-containing gums and mixtures of tobacco with other plant materials, are also known. See for example, the types of smokeless tobacco formulations, ingredients, and processing methodologies set forth in U.S. Pat. No. 1,376,586 to Schwartz; U.S. Pat. No. 4,513,756 to Pittman et al.; U.S. Pat. No. 4,528,993 to Sensabaugh, Jr. et al.; U.S. Pat. No. 4,624,269 to Story et al.; U.S. Pat. No. 4,991,599 to Tibbetts; U.S. Pat. No. 4,987,907 to Townsend; U.S. Pat. No. 5,092,352 to Sprinkle, III et al.; U.S. Pat. No. 5,387,416 to White et al.; U.S. Pat. No. 6,668,839 to Williams; U.S. Pat. No. 6,834,654 to Williams; U.S. Pat. No. 6,953,040 to Atchley et al.; U.S. Pat. No. 7,032,601 to Atchley et al.; and U.S. Pat. No. 7,694,686 to Atchley et al.; US Pat. Pub. Nos. 2004/0020503 to Williams; 2005/0115580 to Quinter et al.; 2006/0191548 to Strickland et al.; 2007/0062549 to Holton, Jr. et al.; 2007/0186941 to Holton, Jr. et al.; 2007/0186942 to Strickland et al.; 2008/0029110 to Dube et al.; 2008/0029116 to Robinson et al.; 2008/0173317 to Robinson et al.; 2008/0209586 to Neilsen et al.; 2009/0065013 to Essen et al.; and 2010/0282267 to Atchley, as well as WO2004/095959 to Arnarp et al., each of which is incorporated herein by reference. It is noted that certain types of oral products that contain tobacco substitutes (or combinations of tobacco and tobacco substitutes) also have been proposed. Certain types of oral products have been employed to contain nicotine, such as those used for nicotine replacement therapy (NRT) types of products (e.g., a pharmaceutical product distributed under the tradename ZONNIC® by Niconovum AB).

Smokeless tobacco product configurations that combine tobacco material with various binders and fillers have been proposed more recently, with example product formats including lozenges, pastilles, gels, extruded forms, and the like. See, for example, the types of products described in US Patent App. Pub. Nos. 2008/0196730 to Engstrom et al.; 2008/0305216 to Crawford et al.; 2009/0293889 to Kumar et al.; 2010/0291245 to Gao et al; 2011/0139164 to Mua et al.; 2012/0037175 to Cantrell et al.; 2012/0055494 to Hunt et al.; 2012/0138073 to Cantrell et al.; 2012/0138074 to Cantrell et al.; 2013/0074855 to Holton, Jr.; 2013/0074856 to Holton, Jr.; 2013/0152953 to Mua et al.; 2013/0274296 to Jackson et al.; 2015/0068545 to Moldoveanu et al.; 2015/0101627 to Marshall et al.; and 2015/0230515 to Lampe et al., each of which is incorporated herein by reference.

Certain types of pouches or sachets have been employed to contain compositions adapted for oral use. See for example, the types of representative smokeless tobacco products, as well as the various smokeless tobacco formulations, ingredients and processing methodologies, referenced in the background art set forth in U.S. Pat. Pub. Nos. 2011/0303511 to Brinkley et al. and 2013/0206150 to Duggins et al.; which are incorporated herein by reference. During use, those pouches or sachets are inserted into the mouth of the user, and water soluble components contained within those pouches or sachets are released as a result of interaction with saliva.

Certain commercially available smokeless tobacco products, such as products commonly referred to as “snus,” comprise ground tobacco materials incorporated within sealed pouches. Representative types of snus products have been manufactured in Europe, particularly in Sweden, by or through companies such as Swedish Match AB (e.g., for brands such as General, Ettan, Goteborgs Rape and Grovsnus); Fiedler & Lundgren AB (e.g., for brands such as Lucky Strike, Granit, Krekt and Mocca); JTI Sweden AB (e.g., for brands such as Gustavus) and Rocker Production AB (e.g., for brands such as Rocker). Other types of snus products have been commercially available in the U.S.A. through companies such as Philip Morris USA, Inc. (e.g., for brands such as Marlboro Snus); U.S. Smokeless Tobacco Company (e.g., for brands such as SKOAL Snus) and R. J. Reynolds Tobacco Company (e.g., for brands such as CAMEL Snus). See also, for example, Bryzgalov et al., 1N1800 Life Cycle Assessment, Comparative Life Cycle Assessment of General Loose and Portion Snus (2005); which is incorporated herein by reference.

Various types of snus products, as well as components for those products and methods for processing components associated with those products, have been proposed. See, for example, U.S. Pat. No. 8,067,046 to Schleef et al. and U.S. Pat. No. 7,861,728 to Holton, Jr. et al.; US Pat. Pub. Nos. 2004/0118422 to Lundin et al.; 2008/0202536 to Torrence et al.; 2009/0025738 to Mua et al.; 2011/0180087 to Gee et al.; 2010/0218779 to Zhuang et al.; 2010/0294291 to Robinson et al.; 2010/0300465 to Zimmermann; 2011/0061666 to Dube et al.; 2011/0303232 to Williams et al.; 2012/0067362 to Mola et al.; 2012/0085360 to Kawata et al.; 2012/0103353 to Sebastian et al. and 2012/0247492 to Kobal et al.; and PCT Pub. Nos. WO 05/063060 to Atchley et al. and WO 08/56135 to Onno; which are incorporated herein by reference. In addition, certain quality standards associated with snus manufacture have been assembled as a so-called GothiaTek standard. Furthermore, various manners and methods useful for the production of snus types of products have been proposed. See, for example, U.S. Pat. No. 4,607,479 to Linden and U.S. Pat. No. 4,631,899 to Nielsen; and US Pat. Pub. Nos. 2008/0156338 to Winterson et al.; 2010/0018539 to Brinkley et al.; 2010/0059069 to Boldrini; 2010/0071711 to Boldrini; 2010/0101189 to Boldrini; 2010/0101588 to Boldrini; 2010/0199601 to Boldrini; 2010/0200005 to Fallon; 2010/0252056 to Gruss et al.; 2011/0284016 to Gunter et al.; 2011/0239591 to Gruss et al.; 2011/0303511 to Brinkley et al.; 2012/0055493 to Novak III et al. and 2012/0103349 to Hansson et al.; and PCT Pub. Nos. WO 2008/081341 to Winterson et al. and WO 2008/146160 to Cecil et al.; which are incorporated herein by reference. Additionally, snus products can be manufactured using equipment such as that available as SB 51-1/T, SBL 50 and SB 53-2/T from Merz Verpackungmaschinen GmBH.

### BRIEF SUMMARY

The present disclosure relates to an oral product adapted for release of a water-soluble component therefrom. The oral products described herein can incorporate binder systems, wherein the binder system possesses or exhibits thermoplastic properties, characteristics or behaviors. For example, the binder system can incorporate at least one type of binding agent that exhibits thermoplastic properties, characteristics or behaviors (e.g., the binding agent can be composed of at least one thermoplastic polymeric material). Additionally, the binder system can incorporate ingredients or materials so as to provide a binding agent that exhibits thermoplastic characteristics (e.g., the binding agent can be composed of at least one thermoplastic binding material in combination with a plasticizer). In various embodiments, the thermoplastic binder system can comprise at least one thermoplastic polymer, at least one additional binding material, and at least one plasticizer.

In certain embodiments, the oral product composition can contain a tobacco-derived product, such as a particulate tobacco material, nicotine, particulate non-tobacco material (e.g., microcrystalline cellulose) that has been treated to contain nicotine and/or flavoring agents, or fibrous plant material (e.g., beet pulp fiber) treated to contain a tobacco extract. In various embodiments, the oral product is a smokeless tobacco product or nicotine replacement therapy product.

The invention includes, without limitation, the following embodiments.

Embodiment 1: A composition, comprising: a thermoplastic binder system in an amount of at least about 5% percent by weight, based on total dry weight of the composition; and at least one of a flavoring agent and an active ingredient; wherein the thermoplastic binder system comprises at least one thermoplastic polymer and at least one plasticizer; wherein the composition is substantially free of unbleached tobacco material, excluding any nicotine component present.

Embodiment 2: A composition according to embodiment 1, wherein the at least one thermoplastic polymer is selected from the group consisting of cellulose ethers, polyvinyl alcohol, polyvinyl acetate, aliphatic polyester, polyvinyl polypyrrolidone, maltodextrin, pullulan, polyethylene oxide, natural gums, and blends thereof.

Embodiment 3: A composition according to any of embodiments 1-2, wherein the at least one thermoplastic polymer is a cellulose ether selected from the group consisting of hydroxypropyl cellulose, ethylcellulose, hydroxypropyl methylcellulose, and blends thereof.

Embodiment 4: A composition according to any of embodiments 1-3, wherein the at least one thermoplastic polymer exhibits thermoplastic behavior at temperatures less than about 100° C.

Embodiment 5: A composition according to any of embodiments 1-4, wherein the thermoplastic binder system comprises at least one of pullulan, gum arabic, and xanthan gum.

Embodiment 6: A composition according to any of embodiments 1-5, wherein the at least one plasticizer comprises a plasticizer selected from the group consisting of glycerin, propylene glycol, polypropylene glycol, polyethylene glycol, sorbitol, maltitol, polyglycitol, erythritol, isomalt, xylitol, mannitol, and blends thereof.

Embodiment 7: A composition according to any of embodiments 1-6, wherein the at least one plasticizer is in the form of a plasticizer blend comprising: at least one of sorbitol, maltitol, and polyglycitol; at least one of erythritol, isomalt, xylitol, and mannitol; and at least one of glycerin, propylene glycol, polypropylene glycol, and polyethylene glycol.

Embodiment 8: A composition according to any of embodiments 1-7, wherein the at least one thermoplastic polymer has a softening temperature, and wherein the at least one plasticizer is capable of lowering the softening temperature by about 20° C. or more.

Embodiment 9: A composition according to any of embodiments 1-8, wherein the active ingredient is selected from the group consisting of a nicotine component, botanicals, nutraceuticals, stimulants, amino acids, vitamins, and cannabinoids.

Embodiment 10: A composition according to any of embodiments 1-9, comprising from about 0.001 to about 10% by weight of a nicotine component, calculated as the free base and based on the total dry weight of the composition.

Embodiment 11: A composition according to any of embodiments 1-10, wherein the composition is substantially free of tobacco material, excluding any nicotine component present.

Embodiment 12: A composition according to any of embodiments 1-11, wherein the thermoplastic binder system is present in an amount of at least about 20% percent by weight, based on total dry weight of the composition.

Embodiment 13: A composition according to any of embodiments 1-12, comprising: about 5 to about 50% by dry weight thermoplastic binding system; about 5 to about 75% by dry weight of filler; about 0.1 to about 5% by dry weight of sweetener; about 0.5 to about 7.5% by dry weight of salt; and about 1 to about 10% by dry weight of one or more active ingredients, flavoring agents, or combinations thereof.

Embodiment 14: A method of forming a composition, comprising: contacting a thermoplastic binder system with at least one of an active ingredient and a flavorant to provide a mixture; subjecting the mixture to an elevated temperature; forming the mixture into a desired shape of an oral product; and cooling the oral product; wherein the thermoplastic binder system comprises at least one thermoplastic polymer and at least one plasticizer; wherein the composition is substantially free of unbleached tobacco material, excluding any nicotine component present.

Embodiment 15: A method according to embodiment 14, wherein the at least one thermoplastic polymer is selected from the group consisting of cellulose ethers, polyvinyl alcohol, polyvinyl acetate, aliphatic polyester, polyvinyl polypyrrolidone, maltodextrin, pullulan, polyethylene oxide, natural gums, and blends thereof.

Embodiment 16: A method according to any of embodiments 14-15, wherein the at least one thermoplastic polymer exhibits thermoplastic behavior at temperatures less than about 100° C.

Embodiment 17: A method according to any of embodiments 14-16, wherein the at least one plasticizer comprises a plasticizer selected from the group consisting of glycerin, propylene glycol, polypropylene glycol, polyethylene glycol, sorbitol, maltitol, polyglycitol, erythritol, isomalt, xylitol, mannitol, and blends thereof.

Embodiment 18: A method according to any of embodiments 14-17, wherein the active ingredient is selected from the group consisting of a nicotine component, botanicals, nutraceuticals, stimulants, amino acids, vitamins, and cannabinoids.

Embodiment 19: A method according to any of embodiments 14-18, wherein the composition is substantially free of tobacco material, excluding any nicotine component present.

Embodiment 20: A method according to any of embodiments 14-19, wherein the thermoplastic binder system is present in an amount of at least about 10% percent by weight, based on total dry weight of the composition.

Embodiment 21: An oral product prepared according to the method of any of embodiments 14-20.

These and other features, aspects, and advantages of the disclosure will be apparent from a reading of the following detailed description. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### DETAILED DESCRIPTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As used in this specification and the claims, the singular forms “a,” “an,” and “the” include plural referents unless the context clearly dictates otherwise.

The disclosure generally provides products configured for oral use. The term “configured for oral use” as used herein means that the product is provided in a form such that during use, saliva in the mouth of the user causes one or more of the components of the mixture (e.g., flavoring agents and/or nicotine) to pass into the mouth of the user. In certain embodiments, the product is adapted to deliver components to a user through mucous membranes in the user's mouth and, in some instances, said component is an active ingredient (including, but not limited to, for example, nicotine) that can be absorbed through the mucous membranes in the mouth when the product is used.

In particular, the disclosure provides products in the form of a mixture of one or more components, including at least one active agent and/or at least one flavorant and a binder system. The active agent(s) and/or flavorant(s), as well as any other suitable ingredients, are processed in the presence of a binder system during production or assembly of the oral product. In certain preferred embodiments, the binder system possesses or exhibits thermoplastic properties, characteristics or behaviors. See, e.g., the oral products and binder systems described in U.S. Pat. Pub. No. 2011/0220130 to Mua et al., which is herein incorporated by reference in its entirety.

### Oral Product Composition

The oral products described herein generally comprise a mixture that is capable of being processed, extruded, and/or molded into a final shape.

### Binding Agents

The composition incorporates a binder system. The binder system may be employed in certain embodiments, in amounts sufficient to provide the desired physical attributes and physical integrity to the mixture. The binder systems disclosed herein include at least one type of binding agent that exhibits thermoplastic properties, characteristics, or behaviors (e.g., the binding agent can include at least one thermoplastic polymer, at least one thermoplastic binding material, and/or at least one plasticizing agent). In some embodiments, the binder system includes ingredients or materials which in combination provide a binding agent that exhibits thermoplastic characteristics. The amount of thermoplastic binder system material employed within embodiments of the oral products described herein typically is at least about 5 percent, at least about 10 percent, and often at least about 20 percent, of the final formed product, on a dry weight basis. The amount of thermoplastic binder system material employed within the mixtures described herein generally is less than about 50 percent, and can be less than about 35 percent, less than about 30 percent, or less than about 25 percent of the final formed product, on a dry weight basis. In some embodiments, the amount of binder system in the mixture can be present in an amount of about 5 to about 50 weight percent, about 10 to about 45 weight percent, or about 20 to about 40 weight percent, based on the total dry weight of the mixture.

The ingredient materials of the thermoplastic binder system can vary. The thermoplastic binder systems described herein include at least one thermoplastic polymer. The term thermoplastic as used herein refers to a property of an ingredient (e.g., a polymeric material) where upon heating, the ingredient softens or melts into a liquid that when cooled, hardens or forms a gel. For example, a polymeric material that comprises thermoplastic properties, characteristics or behaviors softens or melts when exposed to heat, and then returns to its original physical type of condition when cooled (e.g., to about ambient temperature). As such, a thermoplastic binding agent can be contacted with an active agent(s) and/or flavorant(s) and various other ingredients, mixed so as to provide physical contact of the binding agent with those ingredients, heated so as to soften or liquefy the polymeric material of the thermoplastic binder system, and then cooled so as to harden the softened thermoplastic polymeric material (and hence result in an oral product that is formed from the ingredients and that has a desirable physical integrity). For purposes of the present disclosure, desirable thermoplastic polymeric materials useful in the binder systems and oral products described herein generally soften or melt (and hence exhibit thermoplastic characteristics) at temperatures of about 50° C. or greater, about 60° C. or greater, about 70° C. or greater, about 100° C. or greater, about 120° C. or greater, about 150° C. or greater, or about 180° C. or greater. In various embodiments, the thermoplastic polymer can have a melting point of about 200° C. or less, about 300° C. or less, 250° C. or less, about 200° C. or less, about 160° C. or less, about 150° C. or less, about 140° C. or less, about 120° C. or less, about 100° C. or less, or about 90° C. or less. In some embodiments, the thermoplastic polymer can have a melting point in the range of about 100° C. to about 350° C., or about 200° C. to about 300° C.

Example thermoplastic polymers include various polyolefin and polyester materials. In certain embodiments, the thermoplastic polymer of the binder system can be a biodegradable polymer, such as an aliphatic polyester. Example aliphatic polyesters include polyglycolic acid (PGA), polylactic acid (PLA) (e.g., poly(L-lactic acid) or poly(DL-lactic acid)), polyhydroxyalkanoates (PHAs) such as polyhydroxypropionate, polyhydroxyvalerate, polyhydroxybutyrate, polyhydroxyhexanoate, and polyhydroxyoctanoate, polycaprolactone (PCL), polybutylene succinate, polybutylene succinate adipate, and copolymers thereof (e.g., polyhydroxybutyrate-co-hydroxyvalerate (PHBV)). In some embodiments, the thermoplastic polymer can include polyethylene oxide, certain cellulose ethers (e.g., hydroxypropyl cellulose, ethylcellulose and hydroxypropyl methylcellulose), polyvinyl alcohol and polyvinyl acetate.

In certain embodiments, the binder system can include a thermoplastic polymer selected from the group consisting of polyvinyl polypyrrolidone, methylcellulose, maltodextrin, pullulan, certain modified starches and high molecular weight propylene glycols (e.g., propylene glycols having molecular weights above about 4000 Da), and combinations thereof. In certain embodiments, the binder system includes a gum, for example, a natural gum. As used herein, a natural gum refers to polysaccharide materials of natural origin that have binding properties, and which are also useful as a thickening or gelling agents. Representative natural gums derived from plants, which are typically water soluble to some degree, include xanthan gum, guar gum, gum arabic, ghatti gum, gum tragacanth, karaya gum, locust bean gum, gellan gum, and combinations thereof.

In various embodiments of the oral products described herein, a thermoplastic polymer can be present in an amount of at least about 5 percent, at least about 10 percent, or at least about 15 percent of the final formed product, on a dry weight basis. In some embodiments, the amount of thermoplastic polymeric material employed within a representative oral product generally is less than about 30 percent, less than about 25 percent, less than about 20 percent, or less than about 15 percent of the final formed product, on a dry weight basis.

The amount of thermoplastic binding materials employed within a representative processed oral product of the present disclosure can be at least about 5 percent, at least about 10 percent, or at least about 15 percent of the final formed product, on a dry weight basis. The amount of thermoplastic binding material employed within a representative processed oral product of the present disclosure generally can be about 40 percent or less, about 30 percent or less, about 25 percent or less, about 20 percent or less, or about 15 percent or less of the final formed product, on a dry weight basis.

The binder system can incorporate a compound that can be characterized as a plasticizer. For example, the aforementioned thermoplastic binding materials can be combined with, and processed in combination with, at least one plasticizer. In some oral products, a binder system can be employed with a plasticizer blend. In some embodiments, the plasticizer blend can be pre-mixed separate from the mixing with a binder system or other ingredients.

In various embodiments, the plasticizer can be organic non-polymeric materials. For example, the plasticizer can include glycerin, propylene glycol, polyethylene glycol, polypropylene glycol, and combinations thereof. In certain embodiments, the plasticizer can comprise a medium and/or high molecular weight polyol-type compounds. Some example representative polyols include maltitol, sorbitol, isomalt, erythritol, xylitol, mannitol, polyglycitol, and combinations thereof.

In some embodiments, the binder system can include a plasticizer blend. The plasticizer blend can comprise various polyols, such as maltitol, sorbitol, isomalt, erythritol, xylitol, mannitol, and polyglycitol; glycerin; propylene glycol; polyethylene glycol; polypropylene glycol; and combinations thereof. In some plasticizer blends, a syrup or liquid form of a polyol can be employed, for example, maltitol syrup or sorbitol liquid. The syrup or liquid form of the polyol can include at least 20 percent solids, at least 35 percent solids, at least 50 percent solids, at least 60 percent solids, at least 70 percent solids, or at least 75 percent solids.

The amount of plasticizer utilized within the oral product can vary. When employed, the amount of plasticizer present within a representative oral product can be at least about 1 percent, at least about 2 percent, at least about 3 percent, at least about 5 percent, at least about 10 percent, or at least about 15 percent of the final formed product, on a dry weight basis. When employed, the amount of plasticizer employed within a representative processed oral product typically is about 50 percent or less, about 35 percent or less, about 25 percent or less, 15 percent or less, about 10 percent or less, or about 5 percent or less, of the final formed product, on a dry weight basis.

In some embodiments, the plasticizer (including blends thereof) can be prepared separately and then added to a premixed oral product mixture including a binder system as described herein. The plasticizer can be prepared by mixing its granular components with water. The plasticizer can be heated for a period of time and stirred until the plasticizer blend exhibits clear, viscous liquid properties. In some embodiments, the plasticizer can be prepared without the mixing of its granular components with water. By mixing and heating the plasticizer separate from the oral product mixture and binding system, the plasticizer can be heated to a higher temperature without concern of charring and/or scalding the other components of the oral product composition. Upon subjecting the plasticizer to a higher temperature, the plasticizer blend can exhibit plastic-type or visco-elastic properties. The viscosity of such a plasticizer at about 25° C. is typically less than about 25,000 centipoise (cp), and often less than about 15,000 cp. When employed in an oral product, the viscosity of the plasticizer at about 25° C. is about 100 cp or greater, or about 1500 cp or greater. Without being limited by theory, an oral product incorporating a plasticizer having such plastic-like or visco-elastic properties can provide a chewable tobacco product that does not crumble or fall apart in a user's mouth upon light to moderate chewing. Such a chewable tobacco product can change shape in the mouth when lightly to moderately chewed without disintegrating into small particles.

The selection and amount of plasticizer(s) or plasticizer blend can be such that the softening temperature of the thermoplastic binding material is lowered. For example, HPC (KLUCEL) a thermoplastic material, has a melting point of about 370° C., which is lowered to less than 250° C. in the presence of either the plasticizer propylene glycol (PG) or glycerol. HPMC (BENECEL E4M) has a melting point of about 200° C., which is lowered to less than 150° C. in the presence of PG or glycerol. The above involve the inclusion of glycerol or PG at about 10-20% by weight. Without being limited by theory, the plasticizer can act to facilitate hydration, and hence facilitate liquification of the thermoplastic binding material. As such, formation of the oral product at relatively low temperatures can be facilitated (e.g., at a temperature lower than the normal melting point of the thermoplastic binding material). In this way, the binder system within an oral formulation of the present disclosure can lower the operating temperature at which the oral formulation is formed, thereby reducing and/or avoiding the scalding and/or charring of the other materials in the mixture. In addition, the plasticizer can reduce the need for large amounts of moisture, or other liquid carrier materials, that in turn need to be boiled off through drying stages after formation of the oral product.

Thermoplastic polymeric materials, plasticizers, and mixtures of these materials can be processed at the processing and operating conditions described in more detail below. Such thermoplastic polymeric materials and plasticizers typically exhibit thermoplastic characteristics at temperatures below about 140° C., below about 120° C., or below about 100° C.

### Active Ingredient

The mixture may additionally include one or more active ingredients including, but not limited to, a nicotine component, botanical ingredients (e.g., lavender, peppermint, chamomile, basil, rosemary, ginger, cannabis, ginseng, maca, hemp, eucalyptus, rooibos, fennel, citrus, cloves, and tisanes), stimulants (e.g., caffeine and guarana), amino acids (e.g., taurine, theanine, phenylalanine, tyrosine, and tryptophan) and/or pharmaceutical, nutraceutical, and medicinal ingredients (e.g., vitamins, such as B6, B12, and C, and/or cannabinoids, such as tetrahydrocannabinol (THC) and cannabidiol (CBD)). The particular percentages and choice of ingredients will vary depending upon the desired flavor, texture, and other characteristics. Example active ingredients would include any ingredient known to impact one or more biological functions within the body, such as ingredients that furnish pharmacological activity or other direct effect in the diagnosis, cure, mitigation, treatment, or prevention of disease, or which affect the structure or any function of the body of humans or other animals (e.g., provide a stimulating action on the central nervous system, have an energizing effect, an antipyretic or analgesic action, or an otherwise useful effect on the body).

In certain embodiments, the products of the present disclosure can include a nicotinic compound. Various nicotinic compounds, and methods for their administration, are set forth in US Pat. Pub. No. 2011/0274628 to Borschke, which is incorporated herein by reference. As used herein, “nicotinic compound” or “source of nicotine” often refers to naturally-occurring or synthetic nicotinic compound unbound from a plant material, meaning the compound is at least partially purified and not contained within a plant structure, such as a tobacco leaf. Most preferably, nicotine is naturally-occurring and obtained as an extract from a Nicotiana species (e.g., tobacco). The nicotine can have the enantiomeric form S(−)-nicotine, R(+)-nicotine, or a mixture of S(−)-nicotine and R(+)-nicotine. Most preferably, the nicotine is in the form of S(−)-nicotine (e.g., in a form that is virtually all S(−)-nicotine) or a racemic mixture composed primarily or predominantly of S(−)-nicotine (e.g., a mixture composed of about 95 weight parts S(−)-nicotine and about 5 weight parts R(+)-nicotine). Most preferably, the nicotine is employed in virtually pure form or in an essentially pure form. Highly preferred nicotine that is employed has a purity of greater than about 95 percent, more preferably greater than about 98 percent, and most preferably greater than about 99 percent, on a weight basis.

In certain embodiments, a nicotine component may be included in the mixture in free base form, salt form, as a complex, or as a solvate. By “nicotine component” is meant any suitable form of nicotine (e.g., free base or salt) for providing oral absorption of at least a portion of the nicotine present. Typically, the nicotine component is selected from the group consisting of nicotine free base and a nicotine salt. In some embodiments, nicotine is in its free base form, which easily can be adsorbed in for example, a microcrystalline cellulose material to form a microcrystalline cellulose-nicotine carrier complex. See, for example, the discussion of nicotine in free base form in US Pat. Pub. No. 2004/0191322 to Hansson, which is incorporated herein by reference.

In some embodiments, at least a portion of the nicotine can be employed in the form of a salt. Salts of nicotine can be provided using the types of ingredients and techniques set forth in U.S. Pat. No. 2,033,909 to Cox et al. and Perfetti, Beitrage Tabakforschung Int., 12: 43-54 (1983), which are incorporated herein by reference. Additionally, salts of nicotine are available from sources such as Pfaltz and Bauer, Inc. and K&K Laboratories, Division of ICN Biochemicals, Inc. Typically, the nicotine component is selected from the group consisting of nicotine free base, a nicotine salt such as hydrochloride, dihydrochloride, monotartrate, bitartrate, sulfate, salicylate, and nicotine zinc chloride. In some embodiments, the nicotine component or a portion thereof is a nicotine salt with one or more organic acids.

In some embodiments, at least a portion of the nicotine can be in the form of a resin complex of nicotine, where nicotine is bound in an ion-exchange resin, such as nicotine polacrilex, which is nicotine bound to, for example, a polymethacrilic acid, such as Amberlite IRP64, Purolite C115HMR, or Doshion P551. See, for example, U.S. Pat. No. 3,901,248 to Lichtneckert et al., which is incorporated herein by reference. Another example is a nicotine-polyacrylic carbomer complex, such as with Carbopol 974P. In some embodiments, nicotine may be present in the form of a nicotine polyacrylic complex.

Typically, the nicotine component (calculated as the free base) when present, is in a concentration of at least about 0.001% by weight of the mixture, such as in a range from about 0.001% to about 10%. In some embodiments, the nicotine component is present in a concentration from about 0.1% w/w to about 10% by weight, such as, e.g., from about from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10% by weight, calculated as the free base and based on the total weight of the mixture. In some embodiments, the nicotine component is present in a concentration from about 0.1% w/w to about 3% by weight, such as, e.g., from about from about 0.1% w/w to about 2.5%, from about 0.1% to about 2.0%, from about 0.1% to about 1.5%, or from about 0.1% to about 1% by weight, calculated as the free base and based on the total weight of the mixture. These ranges can also apply to other active ingredients noted herein.

### Tobacco Material

In some embodiments, the mixture may include a tobacco material. The tobacco material can vary in species, type, and form. Generally, the tobacco material is obtained from for a harvested plant of the Nicotiana species. Example Nicotiana species include N. tabacum, N. rustica, N. alata, N. arentsii, N. excelsior, N. forgetiana, N. glauca, N. glutinosa, N. gossei, N. kawakamii, N. knightiana, N. langsdorffi, N. otophora, N. setchelli, N. sylvestris, N. tomentosa, N. tomentosiformis, N. undulata, N. x sanderae, N. africana, N. amplexicaulis, N. benavidesii, N. bonariensis, N. debneyi, N. longiflora, N. maritina, N. megalosiphon, N. occidentalis, N. paniculata, N. plumbaginifolia, N. raimondii, N. rosulata, N. simulans, N. stocktonii, N. suaveolens, N. umbratica, N. velutina, N. wigandioides, N. acaulis, N. acuminata, N. attenuata, N. benthamiana, N. cavicola, N. clevelandii, N. cordifolia, N. corymbosa, N. fragrans, N. goodspeedii, N. linearis, N. miersii, N. nudicaulis, N. obtusifolia, N. occidentalis subsp. Hersperis, N. pauciflora, N. petunioides, N. quadrivalvis, N. repanda, N. rotundifolia, N. solanifolia, and N. spegazzinii. Various representative other types of plants from the Nicotiana species are set forth in Goodspeed, The Genus Nicotiana, (Chonica Botanica) (1954); U.S. Pat. No. 4,660,577 to Sensabaugh, Jr. et al.; U.S. Pat. No. 5,387,416 to White et al., U.S. Pat. No. 7,025,066 to Lawson et al.; U.S. Pat. No. 7,798,153 to Lawrence, Jr. and U.S. Pat. No. 8,186,360 to Marshall et al.; each of which is incorporated herein by reference. Descriptions of various types of tobaccos, growing practices and harvesting practices are set forth in Tobacco Production, Chemistry and Technology, Davis et al. (Eds.) (1999), which is incorporated herein by reference.

Nicotiana species from which suitable tobacco materials can be obtained can be derived using genetic-modification or crossbreeding techniques (e.g., tobacco plants can be genetically engineered or crossbred to increase or decrease production of components, characteristics or attributes). See, for example, the types of genetic modifications of plants set forth in U.S. Pat. No. 5,539,093 to Fitzmaurice et al.; U.S. Pat. No. 5,668,295 to Wahab et al.; U.S. Pat. No. 5,705,624 to Fitzmaurice et al.; U.S. Pat. No. 5,844,119 to Weigl; U.S. Pat. No. 6,730,832 to Dominguez et al.; U.S. Pat. No. 7,173,170 to Liu et al.; U.S. Pat. No. 7,208,659 to Colliver et al. and U.S. Pat. No. 7,230,160 to Benning et al.; US Patent Appl. Pub. No. 2006/0236434 to Conkling et al.; and PCT WO2008/103935 to Nielsen et al. See, also, the types of tobaccos that are set forth in U.S. Pat. No. 4,660,577 to Sensabaugh, Jr. et al.; U.S. Pat. No. 5,387,416 to White et al.; and U.S. Pat. No. 6,730,832 to Dominguez et al., each of which is incorporated herein by reference.

The Nicotiana species can, in some embodiments, be selected for the content of various compounds that are present therein. For example, plants can be selected on the basis that those plants produce relatively high quantities of one or more of the compounds desired to be isolated therefrom. In certain embodiments, plants of the Nicotiana species (e.g., Galpao commun tobacco) are specifically grown for their abundance of leaf surface compounds. Tobacco plants can be grown in greenhouses, growth chambers, or outdoors in fields, or grown hydroponically.

Various parts or portions of the plant of the Nicotiana species can be included within a mixture as disclosed herein. For example, virtually all of the plant (e.g., the whole plant) can be harvested, and employed as such. Alternatively, various parts or pieces of the plant can be harvested or separated for further use after harvest. For example, the flower, leaves, stem, stalk, roots, seeds, and various combinations thereof, can be isolated for further use or treatment. In some embodiments, the tobacco material comprises tobacco leaf (lamina). The mixture disclosed herein can include processed tobacco parts or pieces, cured and aged tobacco in essentially natural lamina and/or stem form, a tobacco extract, extracted tobacco pulp (e.g., using water as a solvent), or a mixture of the foregoing (e.g., a mixture that combines extracted tobacco pulp with granulated cured and aged natural tobacco lamina).

In certain embodiments, the tobacco material comprises solid tobacco material selected from the group consisting of lamina and stems. The tobacco that is used for the mixture most preferably includes tobacco lamina, or a tobacco lamina and stem mixture (of which at least a portion is smoke-treated). Portions of the tobaccos within the mixture may have processed forms, such as processed tobacco stems (e.g., cut-rolled stems, cut-rolled-expanded stems or cut-puffed stems), or volume expanded tobacco (e.g., puffed tobacco, such as dry ice expanded tobacco (DIET)). See, for example, the tobacco expansion processes set forth in U.S. Pat. No. 4,340,073 to de la Burde et al.; U.S. Pat. No. 5,259,403 to Guy et al.; and U.S. Pat. No. 5,908,032 to Poindexter, et al.; and U.S. Pat. No. 7,556,047 to Poindexter, et al., all of which are incorporated by reference. In addition, the d mixture optionally may incorporate tobacco that has been fermented. See, also, the types of tobacco processing techniques set forth in PCT WO2005/063060 to Atchley et al., which is incorporated herein by reference.

The tobacco material is typically used in a form that can be described as particulate (i.e., shredded, ground, granulated, or powder form). The manner by which the tobacco material is provided in a finely divided or powder type of form may vary. Preferably, plant parts or pieces are comminuted, ground or pulverized into a particulate form using equipment and techniques for grinding, milling, or the like. Most preferably, the plant material is relatively dry in form during grinding or milling, using equipment such as hammer mills, cutter heads, air control mills, or the like. For example, tobacco parts or pieces may be ground or milled when the moisture content thereof is less than about 15 weight percent or less than about 5 weight percent. Most preferably, the tobacco material is employed in the form of parts or pieces that have an average particle size between 1.4 millimeters and 250 microns. In some instances, the tobacco particles may be sized to pass through a screen mesh to obtain the particle size range required. If desired, air classification equipment may be used to ensure that small sized tobacco particles of the desired sizes, or range of sizes, may be collected. If desired, differently sized pieces of granulated tobacco may be mixed together.

The manner by which the tobacco is provided in a finely divided or powder type of form may vary. Preferably, tobacco parts or pieces are comminuted, ground or pulverized into a powder type of form using equipment and techniques for grinding, milling, or the like. Most preferably, the tobacco is relatively dry in form during grinding or milling, using equipment such as hammer mills, cutter heads, air control mills, or the like. For example, tobacco parts or pieces may be ground or milled when the moisture content thereof is less than about 15 weight percent to less than about 5 weight percent. For example, the tobacco plant or portion thereof can be separated into individual parts or pieces (e.g., the leaves can be removed from the stems, and/or the stems and leaves can be removed from the stalk). The harvested plant or individual parts or pieces can be further subdivided into parts or pieces (e.g., the leaves can be shredded, cut, comminuted, pulverized, milled or ground into pieces or parts that can be characterized as filler-type pieces, granules, particulates or fine powders). The plant, or parts thereof, can be subjected to external forces or pressure (e.g., by being pressed or subjected to roll treatment). When carrying out such processing conditions, the plant or portion thereof can have a moisture content that approximates its natural moisture content (e.g., its moisture content immediately upon harvest), a moisture content achieved by adding moisture to the plant or portion thereof, or a moisture content that results from the drying of the plant or portion thereof. For example, powdered, pulverized, ground or milled pieces of plants or portions thereof can have moisture contents of less than about 25 weight percent, often less than about 20 weight percent, and frequently less than about 15 weight percent.

For the preparation of oral products, it is typical for a harvested plant of the Nicotiana species to be subjected to a curing process. The tobacco materials incorporated within the mixture for inclusion within products as disclosed herein are those that have been appropriately cured and/or aged. Descriptions of various types of curing processes for various types of tobaccos are set forth in Tobacco Production, Chemistry and Technology, Davis et al. (Eds.) (1999). Examples of techniques and conditions for curing flue-cured tobacco are set forth in Nestor et al., Beitrage Tabakforsch. Int., 20, 467-475 (2003) and U.S. Pat. No. 6,895,974 to Peele, which are incorporated herein by reference. Representative techniques and conditions for air curing tobacco are set forth in U.S. Pat. No. 7,650,892 to Groves et al.; Roton et al., Beitrage Tabakforsch. Int., 21, 305-320 (2005) and Staaf et al., Beitrage Tabakforsch. Int., 21, 321-330 (2005), which are incorporated herein by reference. Certain types of tobaccos can be subjected to alternative types of curing processes, such as fire curing or sun curing.

In certain embodiments, tobacco materials that can be employed include flue-cured or Virginia (e.g., K326), burley, sun-cured (e.g., Indian Kurnool and Oriental tobaccos, including Katerini, Prelip, Komotini, Xanthi and Yambol tobaccos), Maryland, dark, dark-fired, dark air cured (e.g., Madole, Passanda, Cubano, Jatin and Bezuki tobaccos), light air cured (e.g., North Wisconsin and Galpao tobaccos), Indian air cured, Red Russian and Rustica tobaccos, as well as various other rare or specialty tobaccos and various blends of any of the foregoing tobaccos.

The tobacco material may also have a so-called “blended” form. For example, the tobacco material may include a mixture of parts or pieces of flue-cured, burley (e.g., Malawi burley tobacco) and Oriental tobaccos (e.g., as tobacco composed of, or derived from, tobacco lamina, or a mixture of tobacco lamina and tobacco stem). For example, a representative blend may incorporate about 30 to about 70 parts burley tobacco (e.g., lamina, or lamina and stem), and about 30 to about 70 parts flue cured tobacco (e.g., stem, lamina, or lamina and stem) on a dry weight basis. Other example tobacco blends incorporate about 75 parts flue-cured tobacco, about 15 parts burley tobacco, and about 10 parts Oriental tobacco; or about 65 parts flue-cured tobacco, about 25 parts burley tobacco, and about 10 parts Oriental tobacco; or about 65 parts flue-cured tobacco, about 10 parts burley tobacco, and about 25 parts Oriental tobacco; on a dry weight basis. Other example tobacco blends incorporate about 20 to about 30 parts Oriental tobacco and about 70 to about 80 parts flue-cured tobacco on a dry weight basis.

Tobacco materials used in the present disclosure can be subjected to, for example, fermentation, bleaching, and the like. If desired, the tobacco materials can be, for example, irradiated, pasteurized, or otherwise subjected to controlled heat treatment. Such treatment processes are detailed, for example, in U.S. Pat. No. 8,061,362 to Mua et al., which is incorporated herein by reference. In certain embodiments, tobacco materials can be treated with water and an additive capable of inhibiting reaction of asparagine to form acrylamide upon heating of the tobacco material (e.g., an additive selected from the group consisting of lysine, glycine, histidine, alanine, methionine, cysteine, glutamic acid, aspartic acid, proline, phenylalanine, valine, arginine, compositions incorporating di- and trivalent cations, asparaginase, certain non-reducing saccharides, certain reducing agents, phenolic compounds, certain compounds having at least one free thiol group or functionality, oxidizing agents, oxidation catalysts, natural plant extracts (e.g., rosemary extract), and combinations thereof. See, for example, the types of treatment processes described in U.S. Pat. Nos. 8,434,496, 8,944,072, and 8,991,403 to Chen et al., which are all incorporated herein by reference. In certain embodiments, this type of treatment is useful where the original tobacco material is subjected to heat in the processes previously described.

In some embodiments, the type of tobacco material is selected such that it is initially visually lighter in color than other tobacco materials to some degree (e.g., whitened or bleached). In certain embodiments, the oral products described herein are substantially free of unbleached tobacco material. Tobacco pulp can be whitened in certain embodiments according to any means known in the art. For example, bleached tobacco material produced by various whitening methods using various bleaching or oxidizing agents and oxidation catalysts can be used. Example oxidizing agents include peroxides (e.g., hydrogen peroxide), chlorite salts, chlorate salts, perchlorate salts, hypochlorite salts, ozone, ammonia, potassium permanganate, and combinations thereof. Example oxidation catalysts are titanium dioxide, manganese dioxide, and combinations thereof. Processes for treating tobacco with bleaching agents are discussed, for example, in U.S. Pat. No. 787,611 to Daniels, Jr.; U.S. Pat. No. 1,086,306 to Oelenheinz; U.S. Pat. No. 1,437,095 to Delling; U.S. Pat. No. 1,757,477 to Rosenhoch; U.S. Pat. No. 2,122,421 to Hawkinson; U.S. Pat. No. 2,148,147 to Baier; U.S. Pat. No. 2,170,107 to Baier; U.S. Pat. No. 2,274,649 to Baier; U.S. Pat. No. 2,770,239 to Prats et al.; U.S. Pat. No. 3,612,065 to Rosen; U.S. Pat. No. 3,851,653 to Rosen; U.S. Pat. No. 3,889,689 to Rosen; U.S. Pat. No. 3,943,940 to Minami; U.S. Pat. No. 3,943,945 to Rosen; U.S. Pat. No. 4,143,666 to Rainer; U.S. Pat. No. 4,194,514 to Campbell; U.S. Pat. Nos. 4,366,823, 4,366,824, and 4,388,933 to Rainer et al.; U.S. Pat. No. 4,641,667 to Schmekel et al.; U.S. Pat. No. 5,713,376 to Berger; U.S. Pat. No. 9,339,058 to Byrd Jr. et al.; U.S. Pat. No. 9,420,825 to Beeson et al.; and U.S. Pat. No. 9,950,858 to Byrd Jr. et al.; as well as in US Pat. App. Pub. Nos. 2012/0067361 to Bjorkholm et al.; 2016/0073686 to Crooks; 2017/0020183 to Bjorkholm; and 2017/0112183 to Bjorkholm, and in PCT Publ. Appl. Nos. WO1996/031255 to Giolvas and WO2018/083114 to Bjorkholm, all of which are incorporated herein by reference. See also, e.g., U.S. patent application Ser. No. 16/226,742 to Mclanahan et al., filed Dec. 20, 2018, which is herein incorporated by reference in its entirety.

In some embodiments, the whitened tobacco material can have an ISO brightness of at least about 50%, at least about 60%, at least about 65%, at least about 70%, at least about 75%, or at least about 80%. In some embodiments, the whitened tobacco material can have an ISO brightness in the range of about 50% to about 90%, about 55% to about 75%, or about 60% to about 70%. ISO brightness can be measured according to ISO 3688:1999 or ISO 2470-1:2016.

In some embodiments, the whitened tobacco material can be characterized as lightened in color (e.g., “whitened”) in comparison to an untreated tobacco material. White colors are often defined with reference to the International Commission on Illumination's (CIE's) chromaticity diagram. The whitened tobacco material can, in certain embodiments, be characterized as closer on the chromaticity diagram to pure white than an untreated tobacco material.

In various embodiments, the tobacco material can be treated to extract a soluble component of the tobacco material therefrom. “Tobacco extract” as used herein refers to the isolated components of a tobacco material that are extracted from solid tobacco pulp by a solvent that is brought into contact with the tobacco material in an extraction process. Various extraction techniques of tobacco materials can be used to provide a tobacco extract and tobacco solid material. See, for example, the extraction processes described in US Pat. Appl. Pub. No. 2011/0247640 to Beeson et al., which is incorporated herein by reference. Other example techniques for extracting components of tobacco are described in U.S. Pat. No. 4,144,895 to Fiore; U.S. Pat. No. 4,150,677 to Osborne, Jr. et al.; U.S. Pat. No. 4,267,847 to Reid; U.S. Pat. No. 4,289,147 to Wildman et al.; U.S. Pat. No. 4,351,346 to Brummer et al.; U.S. Pat. No. 4,359,059 to Brummer et al.; U.S. Pat. No. 4,506,682 to Muller; U.S. Pat. No. 4,589,428 to Keritsis; U.S. Pat. No. 4,605,016 to Soga et al.; U.S. Pat. No. 4,716,911 to Poulose et al.; U.S. Pat. No. 4,727,889 to Niven, Jr. et al.; U.S. Pat. No. 4,887,618 to Bernasek et al.; U.S. Pat. No. 4,941,484 to Clapp et al.; U.S. Pat. No. 4,967,771 to Fagg et al.; U.S. Pat. No. 4,986,286 to Roberts et al.; U.S. Pat. No. 5,005,593 to Fagg et al.; U.S. Pat. No. 5,018,540 to Grubbs et al.; U.S. Pat. No. 5,060,669 to White et al.; U.S. Pat. No. 5,065,775 to Fagg; U.S. Pat. No. 5,074,319 to White et al.; U.S. Pat. No. 5,099,862 to White et al.; U.S. Pat. No. 5,121,757 to White et al.; U.S. Pat. No. 5,131,414 to Fagg; U.S. Pat. No. 5,131,415 to Munoz et al.; U.S. Pat. No. 5,148,819 to Fagg; U.S. Pat. No. 5,197,494 to Kramer; U.S. Pat. No. 5,230,354 to Smith et al.; U.S. Pat. No. 5,234,008 to Fagg; U.S. Pat. No. 5,243,999 to Smith; U.S. Pat. No. 5,301,694 to Raymond et al.; U.S. Pat. No. 5,318,050 to Gonzalez-Parra et al.; U.S. Pat. No. 5,343,879 to Teague; U.S. Pat. No. 5,360,022 to Newton; U.S. Pat. No. 5,435,325 to Clapp et al.; U.S. Pat. No. 5,445,169 to Brinkley et al.; U.S. Pat. No. 6,131,584 to Lauterbach; U.S. Pat. No. 6,298,859 to Kierulff et al.; U.S. Pat. No. 6,772,767 to Mua et al.; and U.S. Pat. No. 7,337,782 to Thompson, all of which are incorporated by reference herein.

Typical inclusion ranges for tobacco materials can vary depending on the nature and type of the tobacco material, and the intended effect on the final mixture, with an example range of up to about 30% by weight (or up to about 20% by weight or up to about 10% by weight or up to about 5% by weight), based on total weight of the mixture (e.g., about 0.1 to about 15% by weight).

In some embodiments, the products of the disclosure can be characterized as completely free or substantially free of tobacco material (other than purified nicotine as an active ingredient). For example, certain embodiments can be characterized as having less than 1% by weight, or less than 0.5% by weight, or less than 0.1% by weight of tobacco material, or 0% by weight of tobacco material.

### Flavoring Agent

As used herein, a “flavoring agent” or “flavorant” is any flavorful or aromatic substance capable of altering the sensory characteristics associated with the oral product. Examples of sensory characteristics that can be modified by the flavoring agent include taste, mouthfeel, moistness, coolness/heat, and/or fragrance/aroma. Flavoring agents may be natural or synthetic, and the character of the flavors imparted thereby may be described, without limitation, as fresh, sweet, herbal, confectionary, floral, fruity, or spicy. Specific types of flavors include, but are not limited to, vanilla, coffee, chocolate/cocoa, cream, mint, spearmint, menthol, peppermint, wintergreen, eucalyptus, lavender, cardamon, nutmeg, cinnamon, clove, cascarilla, sandalwood, honey, jasmine, ginger, anise, sage, licorice, lemon, orange, apple, peach, lime, cherry, strawberry, trigeminal sensates, and any combinations thereof. See also, Leffingwell et al., Tobacco Flavoring for Smoking Products, R. J. Reynolds Tobacco Company (1972), which is incorporated herein by reference. Flavorings also may include components that are considered moistening, cooling or smoothening agents, such as eucalyptus. These flavors may be provided neat (i.e., alone) or in a composite, and may be employed as concentrates or flavor packages (e.g., spearmint and menthol, orange and cinnamon; lime, pineapple, and the like). Representative types of components also are set forth in U.S. Pat. No. 5,387,416 to White et al.; US Pat. App. Pub. No. 2005/0244521 to Strickland et al.; and PCT Application Pub. No. WO 05/041699 to Quinter et al., each of which is incorporated herein by reference. In some instances, the flavoring agent may be provided in a spray-dried form or a liquid form.

The flavoring agent generally comprises at least one volatile flavor component. As used herein, “volatile” refers to a chemical substance that forms a vapor readily at ambient temperatures (i.e., a chemical substance that has a high vapor pressure at a given temperature relative to a nonvolatile substance). Typically, a volatile flavor component has a molecular weight below about 400 Da, and often include at least one carbon-carbon double bond, carbon-oxygen double bond, or both. In one embodiment, the at least one volatile flavor component comprises one or more alcohols, aldehydes, aromatic hydrocarbons, ketones, esters, terpenes, terpenoids, or a combination thereof. Non-limiting examples of aldehydes include vanillin, ethyl vanillin, p-anisaldehyde, hexanal, furfural, isovaleraldehyde, cuminaldehyde, benzaldehyde, and citronellal. Non-limiting examples of ketones include 1-hydroxy-2-propanone and 2-hydroxy-3-methyl-2-cyclopentenone-1-one. Non-limiting examples of esters include allyl hexanoate, ethyl heptanoate, ethyl hexanoate, isoamyl acetate, and 3-methylbutyl acetate. Non-limiting examples of terpenes include sabinene, limonene, gamma-terpinene, beta-farnesene, nerolidol, thuj one, myrcene, geraniol, nerol, citronellol, linalool, and eucalyptol. In one embodiment, the at least one volatile flavor component comprises one or more of ethyl vanillin, cinnamaldehyde, sabinene, limonene, gamma-terpinene, beta-farnesene, or citral. In one embodiment, the at least one volatile flavor component comprises ethyl vanillin.

The amount of flavoring agent utilized in the mixture can vary, but is typically up to about 10 weight percent, and certain embodiments are characterized by a flavoring agent content of at least about 0.1 weight percent, such as about 0.5 to about 10 weight percent, about 1 to about 6 weight percent, or about 2 to about 5 weight percent, based on the total dry weight of the composition.

### Salts

In some embodiments, the composition may further comprise a salt (e.g., alkali metal salts), typically employed in an amount sufficient to provide desired sensory attributes to the composition. Non-limiting examples of suitable salts include sodium chloride, potassium chloride, ammonium chloride, flour salt, and the like. When present, a representative amount of salt is about 0.5 percent by weight or more, about 1.0 percent by weight or more, or at about 1.5 percent by weight or more, but will typically make up about 10 percent or less of the total dry weight of the composition, or about 7.5 percent or less or about 5 percent or less (e.g., about 0.5 to about 5 percent by weight).

### Sweeteners

The composition typically further comprises one or more sweeteners. The sweeteners can be any sweetener or combination of sweeteners, in natural or artificial form, or as a combination of natural and artificial sweeteners. Examples of natural sweeteners include fructose, sucrose, glucose, maltose, mannose, galactose, lactose, stevia, honey, and the like. Examples of artificial sweeteners include sucralose, isomaltulose, maltodextrin, saccharin, aspartame, acesulfame K, neotame and the like. In some embodiments, the sweetener comprises one or more sugar alcohols. Sugar alcohols are polyols derived from monosaccharides or disaccharides that have a partially or fully hydrogenated form. Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates). When present, a representative amount of sweetener may make up from about 0.1 to about 20 percent or more of the of the composition by weight, for example, from about 0.1 to about 1%, from about 1 to about 5%, from about 5 to about 10%, or from about 10 to about 20% of the composition on a dry weight basis, based on the total weight of the composition.

### Filler Component

The composition typically includes at least one filler, which is optionally in particulate form. Such particulate fillers may fulfill multiple functions, such as enhancing certain organoleptic properties such as texture and mouthfeel, enhancing cohesiveness or compressibility of the product, and the like. Generally, the fillers are porous particulate materials and are cellulose-based. For example, suitable particulate fillers are any non-tobacco plant material or derivative thereof, including cellulose materials derived from such sources. Examples of cellulosic non-tobacco plant material include cereal grains (e.g., maize, oat, barley, rye, buckwheat, and the like), sugar beet (e.g., FIBREX® brand filler available from International Fiber Corporation), bran fiber, and mixtures thereof. Non-limiting examples of derivatives of non-tobacco plant material include starches (e.g., from potato, wheat, rice, corn), natural cellulose, and modified cellulosic materials. Additional examples of potential particulate filler components include maltodextrin, dextrose, calcium carbonate, calcium phosphate, lactose, mannitol, xylitol, and sorbitol. Combinations of fillers can also be used.

“Starch” as used herein may refer to pure starch from any source, modified starch, or starch derivatives. Starch is present, typically in granular form, in almost all green plants and in various types of plant tissues and organs (e.g., seeds, leaves, rhizomes, roots, tubers, shoots, fruits, grains, and stems). Starch can vary in composition, as well as in granular shape and size. Often, starch from different sources has different chemical and physical characteristics. A specific starch can be selected for inclusion in the mixture based on the ability of the starch material to impart a specific organoleptic property to composition. Starches derived from various sources can be used. For example, major sources of starch include cereal grains (e.g., rice, wheat, and maize) and root vegetables (e.g., potatoes and cassava). Other examples of sources of starch include acorns, arrowroot, arracacha, bananas, barley, beans (e.g., favas, lentils, mung beans, peas, chickpeas), breadfruit, buckwheat, canna, chestnuts, colacasia, katakuri, kudzu, malanga, millet, oats, oca, Polynesian arrowroot, sago, sorghum, sweet potato, quinoa, rye, tapioca, taro, tobacco, water chestnuts, and yams. Certain starches are modified starches. A modified starch has undergone one or more structural modifications, often designed to alter its high heat properties. Some starches have been developed by genetic modifications, and are considered to be “modified” starches. Other starches are obtained and subsequently modified. For example, modified starches can be starches that have been subjected to chemical reactions, such as esterification, etherification, oxidation, depolymerization (thinning) by acid catalysis or oxidation in the presence of base, bleaching, transglycosylation and depolymerization (e.g., dextrinization in the presence of a catalyst), cross-linking, enzyme treatment, acetylation, hydroxypropylation, and/or partial hydrolysis. Other starches are modified by heat treatments, such as pregelatinization, dextrinization, and/or cold water swelling processes. Certain modified starches include monostarch phosphate, distarch glycerol, distarch phosphate esterified with sodium trimetaphosphate, phosphate distarch phosphate, acetylated distarch phosphate, starch acetate esterified with acetic anhydride, starch acetate esterified with vinyl acetate, acetylated distarch adipate, acetylated distarch glycerol, hydroxypropyl starch, hydroxypropyl distarch glycerol, starch sodium octenyl succinate.

In some embodiments, the particulate filler component is a cellulose material or cellulose derivative. One particularly suitable particulate filler component for use in the products described herein is microcrystalline cellulose (“mcc”). The mcc may be synthetic or semi-synthetic, or it may be obtained entirely from natural celluloses. The mcc may be selected from the group consisting of AVICEL® grades PH-100, PH-102, PH-103, PH-105, PH-112, PH-113, PH-200, PH-300, PH-302, VIVACEL® grades 101, 102, 12, 20 and EMOCEL® grades 50M and 90M, and the like, and mixtures thereof. In one embodiment, the composition comprises mcc as the particulate filler component. The quantity of mcc present in the composition as described herein may vary according to the desired properties.

The amount of particulate filler component can vary, but is typically up to about 75 percent of the material by weight, based on the total dry weight of the composition. A typical range of particulate filler material (e.g., mcc) within the composition can be from about 10 to about 75 percent by total dry weight of the composition, for example, from about 10, about 15, about 20, about 25, or about 30, to about 35, about 40, about 45, or about 50 weight percent (e.g., about 20 to about 50 weight percent or about 25 to about 45 weight percent). In certain embodiments, the amount of particulate filler material is at least about 10 percent by weight, such as at least about 20 percent, or at least about 25 percent, or at least about 30 percent, or at least about 35 percent, or at least about 40 percent, based on the total dry weight of the composition.

In one embodiment, the particulate filler component further comprises a cellulose derivative or a combination of such derivatives. In some embodiments, the composition comprises from about 1 to about 10% of the cellulose derivative by weight, based on the total dry weight of the composition, with certain embodiments comprising about 1 to about 5% by weight of cellulose derivative. In certain embodiments, the cellulose derivative is a cellulose ether (including carboxyalkyl ethers), meaning a cellulose polymer with the hydrogen of one or more hydroxyl groups in the cellulose structure replaced with an alkyl, hydroxyalkyl, or aryl group. Non-limiting examples of such cellulose derivatives include methylcellulose, hydroxypropylcellulose (“HPC”), hydroxypropylmethylcellulose (“HPMC”), hydroxyethyl cellulose, and carboxymethylcellulose (“CMC”). In one embodiment, the cellulose derivative is one or more of methylcellulose, HPC, HPMC, hydroxyethyl cellulose, and CMC. In one embodiment, the cellulose derivative is HPC. In some embodiments, the composition comprises from about 1 to about 3% HPC by weight, based on the total dry weight of the composition.

### Water

The water content of the composition described herein, prior to use by a consumer of the product, may vary according to the desired properties. Typically, the mixture, as present within the product prior to insertion into the mouth of the user, is less than about 60 percent by weight of water, and generally is from about 1 to about 60% by weight of water, for example, from about 5 to about 55, about 10 to about 50, about 20 to about 45, or about 25 to about 40 percent water by weight, including water amounts of at least about 5% by weight, at least about 10% by weight, at least about 15% by weight, and at least about 20% by weight.

### Humectants

In certain embodiments, one or more humectants may be employed in the composition.

Examples of humectants include, but are not limited to, glycerin, propylene glycol, and the like. Where included, the humectant is typically provided in an amount sufficient to provide desired moisture attributes to the composition. Further, in some instances, the humectant may impart desirable flow characteristics to the composition for depositing in a mold. When present, a humectant will typically make up about 5% or less of the dry weight of the composition (e.g., from about 0.5 to about 5% by weight). When present, a representative amount of humectant is about 0.1% to about 1% by weight, or about 1% to about 5% by weight, based on the total dry weight of the composition.

### Buffering Agents

In certain embodiments, the composition of the present disclosure can comprise pH adjusters or buffering agents. Examples of pH adjusters and buffering agents that can be used include, but are not limited to, metal hydroxides (e.g., alkali metal hydroxides such as sodium hydroxide and potassium hydroxide), and other alkali metal buffers such as metal carbonates (e.g., potassium carbonate or sodium carbonate), or metal bicarbonates such as sodium bicarbonate, and the like. Where present, the buffering agent is typically present in an amount less than about 5 percent based on the dry weight of the composition, for example, from about 0.1% to about 5%, such as, e.g., from about 0.75% to about 4%, from about 0.75% to about 3%, or from about 1% to about 2% by weight, based on the total dry weight. Non-limiting examples of suitable buffers include alkali metals acetates, glycinates, phosphates, glycerophosphates, citrates, carbonates, hydrogen carbonates, borates, or mixtures thereof.

### Colorants

A colorant may be employed in amounts sufficient to provide the desired physical attributes to the composition. Examples of colorants include various dyes and pigments, such as caramel coloring and titanium dioxide. The amount of colorant utilized in the composition can vary, but when present is typically up to about 3 weight percent, such as from about 0.1%, about 0.5%, or about 1%, to about 3% by weight, based on the total dry weight.

### Other Additives

Other additives can be included in the disclosed composition. For example, the composition can be processed, blended, formulated, combined and/or mixed with other materials or ingredients. The additives can be artificial, or can be obtained or derived from herbal or biological sources. Examples of further types of additives include thickening or gelling agents (e.g., fish gelatin), emulsifiers, oral care additives (e.g., thyme oil, eucalyptus oil, and zinc), preservatives (e.g., potassium sorbate and the like), disintegration aids, or combinations thereof. See, for example, those representative components, combination of components, relative amounts of those components, and manners and methods for employing those components, set forth in U.S. Pat. No. 9,237,769 to Mua et al., U.S. Pat. No. 7,861,728 to Holton, Jr. et al., US Pat. App. Pub. No. 2010/0291245 to Gao et al., and US Pat. App. Pub. No. 2007/0062549 to Holton, Jr. et al., each of which is incorporated herein by reference. Typical inclusion ranges for such additional additives can vary depending on the nature and function of the additive and the intended effect on the final composition, with an example range of up to about 10% by weight, based on total dry weight (e.g., about 0.1 to about 5% by weight).

The aforementioned additives can be employed together (e.g., as additive formulations) or separately (e.g., individual additive components can be added at different stages involved in the preparation of the final mixture). Furthermore, the aforementioned types of additives may be encapsulated as provided in the final product or mixture. Example encapsulated additives are described, for example, in WO2010/132444 to Atchley, which has been previously incorporated by reference herein.

In some embodiments, any one or more of a filler component, a tobacco material, and the overall oral product described herein can be described as a particulate material. As used herein, the term “particulate” refers to a material in the form of a plurality of individual particles, some of which can be in the form of an agglomerate of multiple particles, wherein the particles have an average length to width ratio less than 2:1, such as less than 1.5:1, such as about 1:1. In various embodiments, the particles of a particulate material can be described as substantially spherical or granular.

The particle size of a particulate material may be measured by sieve analysis. As the skilled person will readily appreciate, sieve analysis (otherwise known as a gradation test) is a method used to measure the particle size distribution of a particulate material. Typically, sieve analysis involves a nested column of sieves which comprise screens, preferably in the form of wire mesh cloths. A pre-weighed sample may be introduced into the top or uppermost sieve in the column, which has the largest screen openings or mesh size (i.e. the largest pore diameter of the sieve). Each lower sieve in the column has progressively smaller screen openings or mesh sizes than the sieve above. Typically, at the base of the column of sieves is a receiver portion to collect any particles having a particle size smaller than the screen opening size or mesh size of the bottom or lowermost sieve in the column (which has the smallest screen opening or mesh size).

In some embodiments, the column of sieves may be placed on or in a mechanical agitator. The agitator causes the vibration of each of the sieves in the column. The mechanical agitator may be activated for a pre-determined period of time in order to ensure that all particles are collected in the correct sieve. In some embodiments, the column of sieves is agitated for a period of time from 0.5 minutes to 10 minutes, such as from 1 minute to 10 minutes, such as from 1 minute to 5 minutes, such as for approximately 3 minutes. Once the agitation of the sieves in the column is complete, the material collected on each sieve is weighed. The weight of each sample on each sieve may then be divided by the total weight in order to obtain a percentage of the mass retained on each sieve. As the skilled person will readily appreciate, the screen opening sizes or mesh sizes for each sieve in the column used for sieve analysis may be selected based on the granularity or known maximum/minimum particle sizes of the sample to be analysed. In some embodiments, a column of sieves may be used for sieve analysis, wherein the column comprises from 2 to 20 sieves, such as from 5 to 15 sieves. In some embodiments, a column of sieves may be used for sieve analysis, wherein the column comprises 10 sieves. In some embodiments, the largest screen opening or mesh sizes of the sieves used for sieve analysis may be 1000 μm, such as 500 μm, such as 400 μm, such as 300 μm.

In some embodiments, any particulate material referenced herein (e.g., filler component, tobacco material, and the overall oral product) can be characterized as having at least 50% by weight of particles with a particle size as measured by sieve analysis of no greater than about 1000 μm, such as no greater than about 500 μm, such as no greater than about 400 μm, such as no greater than about 350 μm, such as no greater than about 300 μm. In some embodiments, at least 60% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 70% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 80% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 90% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 95% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, at least 99% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm. In some embodiments, approximately 100% by weight of the particles of any particulate material referenced herein have a particle size as measured by sieve analysis of no greater than about 1000 μm, such as no greater than about 500 μm, such as no greater than about 400 μm, such as no greater than about 350 μm, such as no greater than about 300 μm.

In some embodiments, at least 50% by weight, such as at least 60% by weight, such as at least 70% by weight, such as at least 80% by weight, such as at least 90% by weight, such as at least 95% by weight, such as at least 99% by weight of the particles of any particulate material referenced herein have a particle size as measured by sieve analysis of from about 0.01 μm to about 1000 μm, such as from about 0.05 μm to about 750 μm, such as from about 0.1 μm to about 500 μm, such as from about 0.25 μm to about 500 μm. In some embodiments, at least 50% by weight, such as at least 60% by weight, such as at least 70% by weight, such as at least 80% by weight, such as at least 90% by weight, such as at least 95% by weight, such as at least 99% by weight of the particles of any particulate material referenced herein have a particle size as measured by sieve analysis of from about 10 μm to about 400 μm, such as from about 50 μm to about 350 μm, such as from about 100 μm to about 350 μm, such as from about 200 μm to about 300 μm.

### Preparation of the Oral Product

The manner by which the various components of the composition can be combined may vary. The various components of the formulation may be contacted, combined, or mixed together in conical-type blenders, mixing drums, ribbon blenders, or the like. As such, the overall mixture of various components (i.e., the active ingredient(s) and/or flavorant(s), the thermoplastic binder system, and any other ingredients) may be relatively uniform in nature. See also, for example, the types of methodologies set forth in U.S. Pat. No. 4,148,325 to Solomon et al.; U.S. Pat. No. 6,510,855 to Korte et al.; and U.S. Pat. No. 6,834,654 to Williams, each of which is incorporated herein by reference. The components of the oral product formulation can be brought together in admixture using any mixing technique or equipment known in the art. Various components noted herein, which may be in liquid or dry solid form, can be admixed in a pretreatment step prior to mixture with any remaining components of the composition, or the mixture components can be simply mixed all together. Any mixing method that brings the oral composition ingredients into intimate contact can be used. A mixing apparatus featuring an impeller or other structure capable of agitation is typically used. Example mixing equipment includes casing drums, conditioning cylinders or drums, liquid spray apparatus, ribbon blenders, mixers available as FKM130, FKM600, FKM1200, FKM2000 and FKM3000 from Littleford Day, Inc., Plough Share types of mixer cylinders, and the like.

The manner that the oral product is formed can vary, and various methods for forming processed oral products or for producing processed oral products will be readily apparent to those skilled in the art of oral product manufacture. For example, suitably heated oral formulations incorporating thermoplastic binder systems and having a pourable, formable or generally liquid character can be cast into a sheet-like form and cooled to yield a sheet-like oral product. As another example, suitably heated oral product formulations incorporating thermoplastic binder systems can be molded into a desired shape and cooled. Example shapes of formed oral products include pill, tablet, sphere, sheet, film, coin, cube, bead, ovoid, obloid, bean, cylinder, stick, and rod. As yet another example, oral product formulations incorporating thermoplastic binder systems can be extruded from heated extrusion devices and cooled. As another example, oral product formulations incorporating a binder system and a pre-mixed plasticizer blend can be extruded from heated extrusion devices and cooled.

The oral product can be manufactured using hot melt extrusion techniques. As such, processed oral product formulations can be subjected to sufficient heat during formation of oral product to eliminate the need for any additional heat treatment steps. For example, flashing off the moisture from a processed oral product formulation during a hot melt extrusion process can eliminate any need for further heating, drying, or the like.

Processed oral product formulations, such as multi-layered pellets, can be manufactured using a wide variety of extrusion techniques. For example, multi-layered pellets can be manufactured using co-extrusion techniques (e.g., using twin screw extruders). In such a situation, successive wet or dry components or component mixtures can be placed within separate extrusion hoppers. Steam, gases (e.g., ammonia, air, carbon dioxide, and the like), plasticizers (e.g., glycerin or propylene glycol), and a plasticizer blend can be injected into the extruder barrel as each dry mix is propelled, plasticized, and cooked. As such, the various components are processed so as to be very well mixed, and hence, come in complete contact with each other. For example, the contact of components is such that individual components can be well embedded in the extrusion matrix or extrudate. See, for example, U.S. Pat. No. 4,821,749 to Toft et al., which is incorporated herein by reference. Multilayered materials can have the general form of films, and alternatively, multi-layered generally spherical materials can possess various layers extending from the inside outward.

Some shapes of oral products, such as rods or cubes, can be formed by first extruding the material through a die having the desired cross-section (e.g., round or square) and then optionally cutting the extruded material into desired lengths. For example, extruder equipment comprising single or multiple screw extruders can by employed to form the shape of a product according to the present disclosure. Some extruders can comprise twin screw extruders which comprise co-rotating twin screw extruders. Various screw configurations can be employed. For example, screws having combinations of elements for feeding, mixing, pumping, shearing, and the like, can be selected as desired for optimum results. Screws having sections or elements which provide relatively large output capacities, which have interrupted or nonconjugated flights, or which are “counterflighted” or “reversing” also can be employed. Typical screw elements as well as screws having combinations of such elements are available from extruder manufacturers. An extruder can provide a barrel for heating of the materials which are introduced within the extruder. The extruder barrel can comprise multiple barrel zones. The conditions of the extrusion set-up can vary, for example, in modifying feed rate of dry blend ingredients, feed rate of wet blend ingredients, screw RPM setting, temperature conditions, aperture die dimensions, barrel zone entry point for certain ingredients, as well as additional parameters.

Techniques and equipment for extruding oral product materials have been set forth in U.S. Pat. No. 3,098,492 to Wursburg; U.S. Pat. No. 4,874,000 to Tamol et al.; U.S. Pat. No. 4,880,018 to Graves et al.; U.S. Pat. No. 4,989,620 to Keritsis et al.; U.S. Pat. No. 5,072,744 to Luke et al.; U.S. Pat. No. 5,829,453 to White et al.; and U.S. Pat. No. 6,182,670 to White et al.; each of which is incorporated herein by reference. Example extrusion equipment suitable for use include industrial pasta extruders such as Model TP 200/300 available from Emiliomiti, LLC of Italy.

In some methods that can be used to produce oral products, a portion of the ingredients can be mixed and fed into the extruder. Additional ingredients can be introduced to the extruder at a different point or barrel zone as compared to other ingredients. For example, a pre-mixed subgroup of ingredients can be fed into a first barrel zone of the extruder to contact a dry mix of other ingredients. A hopper or plurality of hoppers can be employed to facilitate the input of various ingredients into an extruder.

Processed sheet-like materials can be prepared by applying the oral product composition onto a moving belt and passing the moving belt through a nip formed by opposing rollers, followed by cutting the sheet into desired lengths. Alternatively, the oral product can be over-coated with powdered or liquid coating. Furthermore, if desired, the surface of the oral product can be embossed or printed.

### EXAMPLES

The present invention can be more fully illustrated by the following examples, which are set forth to illustrate some embodiments of the present invention and are not to be construed as limiting thereof. All weight percentages are expressed on a dry weight basis, meaning water content is excluded, unless otherwise indicated.

### Example 1

### Binder System Formulations

Tables A-M provide various embodiments of a binder system that can be incorporated into a composition for use in an oral product representative of the present disclosure. The binder system prepared according to the recipes shown in Tables A-M can be used within the oral product formulations found in the examples described hereinafter.

Each batch of binder system is prepared by mixing the components in a commercially available Kitchen Aid mixer for about 3 to 5 minutes.

TABLE A

| Batch 1 Ingredients | wt % | g |
|---|---|---|
| hydroxypropyl | 70 | 350 |
| methylcellulose | | |
| Propylene Glycol | 30 | 150 |
| Total ingredients: | 100% | 500 |

TABLE B

| Batch 2 Ingredients | wt % | g |
|---|---|---|
| hydroxypropyl | 42 | 210 |
| methylcellulose | | |
| Propylene Glycol | 28 | 140 |
| Water | 30 | 150 |
| Total ingredients: | 100% | 500 |

TABLE C

| Batch 3 Ingredients | wt % | g |
|---|---|---|
| Xanthan | 42 | 210 |
| Maltodextrin | 28 | 140 |
| Propylene Glycol | 30 | 150 |
| Total ingredients: | 100% | 500 |

TABLE D

| Batch 4 Ingredients | wt % | g |
|---|---|---|
| Xanthan | 29.4 | 147 |
| Maltodextrin | 19.6 | 98 |
| Propylene Glycol | 21 | 105 |
| Water | 30 | 150 |
| Total ingredients: | 100% | 500 |

TABLE E

| Batch 5 Ingredients | wt % | g |
|---|---|---|
| Gum Arabic | 42 | 210 |
| Propylene Glycol | 28 | 140 |
| Water | 30 | 150 |
| Total ingredients: | 100% | 500 |

TABLE F

| Batch 6 Ingredients | wt % | g |
|---|---|---|
| Xanthan gum | 21.7 | 108.5 |
| Gum Arabic | 33.6 | 168 |
| polyvinylpyrrolidone | 14.7 | 73.5 |
| Propylene Glycol | 30 | 150 |
| Total ingredients: | 100% | 500 |

TABLE G

| Batch 7 Ingredients | wt % | g |
|---|---|---|
| Gum Arabic | 60 | 300 |
| Maltodextrin | 15 | 75 |
| Propylene Glycol | 25 | 125 |
| Total ingredients: | 100% | 500 |

TABLE H

| Batch 8 Ingredients | wt % | g |
|---|---|---|
| Maltodextrin | 73 | 365 |
| Propylene Glycol | 27 | 135 |
| Total ingredients: | 100% | 500 |

TABLE I

| Batch 9 Ingredients | wt % | g |
|---|---|---|
| Modified Starch | 75 | 375 |
| Propylene Glycol | 25 | 125 |
| Total ingredients: | 100% | 500 |

TABLE J

| Batch 10 Ingredients | wt % | g |
|---|---|---|
| Modified Starch | 65 | 325 |
| Maltodextrin | 10 | 50 |
| Propylene Glycol | 25 | 125 |
| Total ingredients: | 100% | 500 |

TABLE K

| Batch 11 Ingredients | wt % | g |
|---|---|---|
| Pullulan | 42 | 210 |
| Maltodextrin | 28 | 140 |
| Propylene Glycol | 30 | 150 |
| Total ingredients: | 100% | 500 |

TABLE L

| Batch 12 Ingredients | wt % | g |
|---|---|---|
| Pullulan | 29.4 | 147 |
| Maltodextrin | 19.6 | 98 |
| Propylene Glycol | 21 | 105 |
| Water | 30 | 150 |
| Total ingredients: | 100% | 500 |

TABLE M

| Batch 13 Ingredients | wt % | g |
|---|---|---|
| Pullulan | 21.7 | 108.5 |
| Gum Arabic | 33.6 | 168 |
| polyvinylpyrrolidone | 14.7 | 73.5 |
| Propylene Glycol | 30 | 150 |
| Total ingredients: | 100% | 500 |

### Example 2

Oral product formulations for use as a stick type of an oral product as set forth in Table 1 are provided in the following manner.

Except for glycerin, all dry ingredients, in powder form, are added together with a batch formulation and thoroughly mixed. Each batch formulation is prepared by mixing propylene glycol and/or water with a binder in a Kitchen Aid mixer for about 5 minutes, before being mixed with other dry ingredients. The nicotine can be added to the binder system before adding to the dry ingredients. Each mixed formulation is then commuted into the hopper of an extruder. The extruder is operated at a temperature of about 65° C. to 100° C., and the screw speed is set at 60 rpm. The formulation is fed through the extruder and 3.5 mm long sticks are prepared.

TABLE 1

| Ingredients | Weight % |
|---|---|
| Nicotine | 1-5% |
| Salt | 0.5-7.5 |
| Buffer | 0.1-5 |
| Sweetener | 0.1-5 |
| Colorant | 0.1-3 |
| Filler | 5-75 |
| Flavorant | 0.1-10 |
| Other Additive(s) | 0.1-20 |
| Binder System | 20-50 |
| (One of Table A-M) | |
| Total ingredients: | 100 |

### Example 3

Example 2 is generally repeated, but a sheet type product is formed having the general formula of Table 1. All ingredients are added together and thoroughly mixed before being commuted into an extruder. The extruder is set at a screw speed of 45-60 rpm and the barrel sections and die operated at temperatures of about 75-100° C. A sheeting die is employed. The formulation is extruded into a continuous sheet that is about 0.85 mm thick. The sheet is extruded onto a conveyor belt and transferred through a cooling tunnel. The sheet is further reduced in thickness by passage through pin rolls, and is flattened into a sheet having a thickness of less than 0.4 mm. The sheet is finally cut into bite-size strip pieces of various shapes (e.g., pieces of about 2 cm by 2 cm).

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A composition, comprising:
a thermoplastic binder system in an amount of at least about 5% percent by weight, based on total dry weight of the composition; and
at least one of a flavoring agent and an active ingredient;
wherein the thermoplastic binder system comprises at least one thermoplastic polymer and at least one plasticizer;
wherein the composition is substantially free of unbleached tobacco material, excluding any nicotine component present.

2. The composition of claim 1, wherein the at least one thermoplastic polymer is selected from the group consisting of cellulose ethers, polyvinyl alcohol, polyvinyl acetate, aliphatic polyester, polyvinyl polypyrrolidone, maltodextrin, pullulan, polyethylene oxide, natural gums, and blends thereof.

3. The composition of claim 1, wherein the at least one thermoplastic polymer is a cellulose ether selected from the group consisting of hydroxypropyl cellulose, ethylcellulose, hydroxypropyl methylcellulose, and blends thereof.

4. The composition of claim 1, wherein the at least one thermoplastic polymer exhibits thermoplastic behavior at temperatures less than about 100° C.

5. The composition of claim 1, wherein the thermoplastic binder system comprises at least one of pullulan, gum arabic, and xanthan gum.

6. The composition of claim 1, wherein the at least one plasticizer comprises a plasticizer selected from the group consisting of glycerin, propylene glycol, polypropylene glycol, polyethylene glycol, sorbitol, maltitol, polyglycitol, erythritol, isomalt, xylitol, mannitol, and blends thereof.

7. The composition of claim 1, wherein the at least one plasticizer is in the form of a plasticizer blend comprising: at least one of sorbitol, maltitol, and polyglycitol; at least one of erythritol, isomalt, xylitol, and mannitol; and at least one of glycerin, propylene glycol, polypropylene glycol, and polyethylene glycol.

8. The composition of claim 1, wherein the at least one thermoplastic polymer has a softening temperature, and wherein the at least one plasticizer is capable of lowering the softening temperature by about 20° C. or more.

9. The composition of claim 1, wherein the active ingredient is selected from the group consisting of a nicotine component, botanicals, nutraceuticals, stimulants, amino acids, vitamins, and cannabinoids.

10. The composition of claim 1, comprising from about 0.001 to about 10% by weight of a nicotine component, calculated as the free base and based on the total dry weight of the composition.

11. The composition of claim 1, wherein the composition is substantially free of tobacco material, excluding any nicotine component present.

12. The composition of claim 1, wherein the thermoplastic binder system is present in an amount of at least about 20% percent by weight, based on total dry weight of the composition.

13. The composition of claim 1, comprising:
about 5 to about 50% by dry weight thermoplastic binding system;
about 5 to about 75% by dry weight of filler;
about 0.1 to about 5% by dry weight of sweetener;
about 0.5 to about 7.5% by dry weight of salt; and
about 1 to about 10% by dry weight of one or more active ingredients, flavoring agents, or combinations thereof.

14. A method of forming a composition, comprising:
contacting a thermoplastic binder system with at least one of an active ingredient and a flavorant to provide a mixture;
subjecting the mixture to an elevated temperature;
forming the mixture into a desired shape of an oral product; and
cooling the oral product;
wherein the thermoplastic binder system comprises at least one thermoplastic polymer and at least one plasticizer;
wherein the composition is substantially free of unbleached tobacco material, excluding any nicotine component present.

15. The method of claim 14, wherein the at least one thermoplastic polymer is selected from the group consisting of cellulose ethers, polyvinyl alcohol, polyvinyl acetate, aliphatic polyester, polyvinyl polypyrrolidone, maltodextrin, pullulan, polyethylene oxide, natural gums, and blends thereof.

16. The method of claim 14, wherein the at least one thermoplastic polymer exhibits thermoplastic behavior at temperatures less than about 100° C.

17. The method of claim 14, wherein the at least one plasticizer comprises a plasticizer selected from the group consisting of glycerin, propylene glycol, polypropylene glycol, polyethylene glycol, sorbitol, maltitol, polyglycitol, erythritol, isomalt, xylitol, mannitol, and blends thereof.

18. The method of claim 14, wherein the active ingredient is selected from the group consisting of a nicotine component, botanicals, nutraceuticals, stimulants, amino acids, vitamins, and cannabinoids.

19. The method of claim 14, wherein the composition is substantially free of tobacco material, excluding any nicotine component present.

20. The method of claim 14, wherein the thermoplastic binder system is present in an amount of at least about 10% percent by weight, based on total dry weight of the composition.
